(19) United States
(12) Patent Application Publication
(10) Pub. No.: US 2025/0388871 A1
(43) Pub. Date: Dec. 25, 2025

(54) EXTRACELLULAR MATRIX (ECM)-EMBEDDED VASCULAR CHANNEL-ON-CHIP, AIRWAY-ON-A-CHIP AND METHODS OF MAKING SAME

(71) Applicant: UNIVERSITY OF PITTSBURGH-OF THE COMMONWEALTH SYSTEM OF HIGHER EDUCATION, PITTSBURGH, PA (US)

(72) Inventors: KAMBEZ HAJIPOURAN BENAM, MARS, PA (US); XUAN QUOC VO, PITTSBURGH, PA (US)

(73) Assignee: UNIVERSITY OF PITTSBURGH-OF THE COMMONWEALTH SYSTEM OF HIGHER EDUCATION, PITTSBURGH, PA (US)

(21) Appl. No.: 18/873,441

(22) PCT Filed: Sep. 15, 2023
(86) PCT No.: PCT/US2023/032847

§ 371 Date: Dec. 10, 2024

Related U.S. Application Data
(60) Provisional application No. 63/375,754, filed on Sep. 15, 2022.
(60) Provisional application No. 63/525,474, filed on Jul. 7, 2023.

(51) Int. Cl. C12N 5/071; C12M 1/12; C12M 3/00

(52) U.S. Cl. CPC C12N 5/0697; C12M 21/08; C12M 25/02; C12N 2513/00; C12N 2533/40; C12N 2533/56; C12N 2533/90

(57) ABSTRACT

An ECM-embedded vascular channel-on-a-chip includes an outer case having an internal chamber, an ECM provided within the chamber, the ECM having a cross-sectionally rounded vascular channel having a first end and a second end opposite the first end, wherein an immer surface of the channel is lined with barrier-forming endothelial cells, and an inlet conduit coupled to the first end of the channel through a first side of the outer casc and an outlet conduit coupled to the second end of the channel through a second side of the outer case. Also, an airway-on-a-chip includes an airway lumen including a porous membrane, wherein mucociliated airway epithelial cells are provided on the porous membrane. The airway-on-a-chip has an ECM with embedded stromal cells. The ECM has a cross-sectionally rounded vascular channel, and the inner surface of the channel is lined with barrier-forming endothelial cells.

### STATEMENT OF GOVERNMENT INTEREST

This invention was made with government support under grant # HHS75F40121C00039 awarded by the Food and Drug Administration (FDA) and grant #R01HL159494 awarded by the National Institutes of Health (NIH). The government has certain rights in the invention.

### FIELD OF THE INVENTION

The present invention pertains to microphysiological systems, and, in particular, to an extracellular matrix (ECM)-embedded vascular channel-on-a-chip, a next generation airway-on-a-chip, and methods of making an ECM-embedded vascular channel-on-a-chip and/or an airway-on-a-chip.

### BACKGROUND OF THE INVENTION

Organs-on-Chips, commonly referred to as microphysiological systems (MPS), are biomimetic, microfluidic, cell culture devices that contain living tissue cells arranged to simulate tissue-and organ-level physiology, and have potentially emerged as powerful alternatives to animals and 2D culture models for preclinical research and trials. These devices contain continuously perfused hollow microchannels and/or chambers inhabited by living tissue cells arranged to simulate organ-level physiology. By recapitulating the multicellular architectures, tissue-tissue interfaces, chemical gradients, and mechanical cues, these devices produce levels of tissue and organ functionality not possible with conventional 2D or 3D culture systems. They also enable high-resolution, real-time imaging, and in vitro analysis of biochemical, genetic, and metabolic activities of living human cells in a functional tissue and organ context.

To reproduce diverse functions, pathophysiology, and dynamic cellular and biomechanical responses observed in blood vessels, extensive efforts have been made to emulate vasculature in Organs-on-Chips. These can be divided into (I) bottom-up efforts on creating microvascular networks within hydrogels, and (II) top-down efforts focused on developing well-defined perfusable vascular lumens-on-chip.

Approach I relies on the self-organization property of endothelial cells (EnCs), alone or in combination with other co-cultured cells, to form three-dimensional microvascular networks. While helpful in modeling vasculature, the network of blood vessels in this strategy either lacks perfusion or the vascular flow occurs, but the barrier function is not fully maintained throughout the established cellular network. Importantly, given the self-assembly nature of this method, exact control over vascular tube diameter, length, branching, or other geometrical features are often very limited.

Approach II relies on seeding vascular EnCs in pre-formed hollow tubes embedded within a hydrogel or microfluidic lumens. Coating, stamping, casting, 3D-bioprinting, and viscous fingering are the most widely used methods in this approach. Despite their potential for vascularized tissue modeling, these methods have several drawbacks. For instance, for coating, the inner surfaces of a microfabricated channel are coated with extracellular matrix (ECM) protein(s) prior to EnC seeding. In the majority of cases, coating is performed on device lumens with rectangular or square cross-sections rather than in vivo-like fully circular/oval cross-sections. Importantly, the thin layer of ECM coating is not always maintained throughout the cell culture and, as such, EnCs are exposed to supra-physiologically stiff non-biological material (such as polydimethylsiloxane (PDMS), glass, or polymethyl methacrylate (PMMA)) used in device fabrication. Similarly, a stamping approach typically generates square, rectangular, or trapezoidal cross-sectional vascular channels. Moreover, while it is possible to generate ECM-embedded vascular channels with this method, it is difficult to incorporate the vasculature with other components of the chip, such as a live airway lumen in a Lung-on-a-Chip for downstream applications. The casting and bioprinting methods, while offering the ability to generate rounded blood vessels in vitro, have notable challenges in their utility and broader adaptation. Casting, by which a lumenized endothelial compartment is formed within a hydrogel, often (1) utilizes fragile materials, such as PDMS rods to cast vascular channels, that have limited length and commonly require pre-treatment to render them hydrophobic, and (2) suffers medium-to-high failure rates on adaptation and reproduction. In addition, even when using more rigid materials for lumen formation, such as stainless-steel needles, there is a high likelihood of hydrogel pull-out on needle removal, and thus channel collapse and device failure. Bioprinting is a more elaborate process and relies on removing sacrificial bio-inks (such as carbohydrate glass, pluronic acid, or wax) cast as a 3D lattice from polymerized matrix surrounding them. While this is suitable for fabrication of relatively thick tissues and geometrically complex structures, the uniformity and smoothness of the vascular network is limited by the printing resolution as well as the deformation of the printed bio-ink. Additionally, 3D-bioprinting of vasculature is only possible with a limited pool of materials that can be used as sacrificial bio-ink; these must be easily dissolvable, either thermally or chemically, to be evacuated following scaffold ECM polymerization. Viscous fingering uses high pressure to flush out ECM hydrogel filled in a small channel to create vascular lumens. While it is practical to create rounded vascular channels, viscous fingering is only capable of creating channels surrounded by a thin ECM layer, ˜100 μm thick, comparable to that in the coating method. Moreover, it is challenging to change the channel geometries such as its length and diameter as it requires a whole adjustment on the optimal applied pressure. In addition, this method often suffers high level of variability (in precision of channel dimensions) between independent experiments and different users.

The complexity of these approaches, their inherent variabilities, and the discussed drawbacks have partly hampered their widespread adaptation and application.

Organs-on-Chips technology has been adapted to recapitulate human lung pathophysiology (airways and alveoli) in a set of devices that are commonly referred to as Lung-on-a-Chip. These devices consist of two juxtaposed channels (an apical channel and a basal channel) that are separated by a semi-porous membrane. The membrane is made of either polyethylene terephthalate (PET, polyester) or polydimethylsiloxane (PDMS). When modeling conducting airways, airway basal epithelial cells are added to the apical (airway) channel and cells are allowed to mucociliary differentiate under ALI into ciliated cells. Club cells, mucin cells and retain some population of basal cells. The apical channel is connected to surrounding air exposing the airway cells to air during ALI. Culture media are flowed through the basal (vascular) channel to supply nutrition for the airway cells on top through the semi-porous membrane. This setting provides air-liquid interface (ALI) for airway cells. Endothelial vascular cells sometimes are cultured on the undersurface of the membrane (the top surface of the basal channel) to replicate vasculature.

However, the existing Lung-on-a-Chip devices suffer multiple key limitations discussed below. First, endothelial cells, when present, are cultured directly on the undersurface of the semi-porous membrane (and sides of the bottom vascular channel). In other words, endothelial cells in these devices do not interact with real three-dimensional ECM that physiologically surrounds vasculature in the sub-epithelial space of our lungs. Also, a number of device preparations require temporary incubation of microfluidic channels prior to cell seeding with a matrix protein (e.g., collagen, fibronectin). However, these (i) are temporary and short-lived—i.e., are often degraded over time in culture and disappear, (ii) are very thin (often <10 μm) as opposed to 100-1,000 μm seen in vivo, and (iii) do not offer ability to finely regulate matrix rheology. As such, endothelial cells, even in these settings, end up being in close contact with highly stiff structures (the membrane and the device material which are often PET or PDMS), that is not physiological. Second, none of these devices have been able to integrate ECM and ECM-embedded stromal cells as seen in vivo in addition to mucociliated airway epithelia, vascular endothelial cells, and circulating immune cells (if any). As such, they lack physiologically relevant architectural complexity, three-dimensionality, and multi-cellularity. Third, the blood vessel channel in existing Lung-on-a-Chip devices is “square” or “rectangular” in shape—i.e., not physiologically relevant in terms of geometry, and produces a range of—i.e., non-homogenous and non-uniform, vascular shear stress on the endothelial cells. In other words, the sharp corners of these (square-/rectangle-shaped) vascular channels experience a different shear stress compared with flat parts. Importantly, in human bodies, blood vessels are rounded or oval shaped. Fourth, the airway lumen is also “square” or “rectangular” in shape in most of these devices, while physiologically human lung conducting airways are rounded on their cross-section. Recently, U.S. Pat. No. 11,499,128 describes a semi-circular airway lumen for Lung-on-a-Chip devices. But, the semi-porous membrane on top of which epithelial cells are cultured remain flat.

### SUMMARY OF THE INVENTION:

In one embodiment, an ECM-embedded vascular channel-on-a-chip is provided. The vascular channel-on-a-chip includes an outer case having an internal chamber, an ECM provided within the chamber, the ECM having a cross-sectionally rounded vascular channel having a first end and a second end opposite the first end, wherein an inner surface of the channel is lined with barrier-forming endothelial cells, and an inlet conduit coupled to the first end of the channel through a first side of the outer case and an outlet conduit coupled to the second end of the channel through a second side of the outer case.

In another embodiment, a method of making an ECM-embedded vascular channel-on-a-chip is provided. The method includes providing an outer case having an internal chamber, providing an inlet conduit through a first side of the outer case and providing an outlet conduit through a second side of the outer case, the inlet conduit and the outlet conduit each being in fluid communication with the chamber, inserting a tubing member into the chamber through at least one of the inlet conduit and the outlet conduit, casting an ECM hydrogel into the chamber and around the tubing member through a casting port provided in the outer case, polymerizing the ECM hydrogel within the chamber, withdrawing the tubing member from the outer case to leave a vascular channel in the polymerized ECM hydrogel, inserting a tube into the vascular channel, stabilizing the vascular channel with the inserted tube, removing the tube from the vascular channel, and cellularizing the vascular channel to form a layer of endothelial cells on an inner surface of the channel.

In still another embodiment, a Next-Generation-Airway-on-a-Chip (“Next-Gen Airway-Chip”) is provided that includes an outer case having an internal chamber, an airway lumen provided within the outer case, wherein the airway lumen includes a porous membrane, and wherein mucociliated airway epithelial cells are provided on the porous membrane, and an ECM is provided within the chamber, wherein stromal cells are embedded within the ECM. The ECM has a cross-sectionally rounded vascular channel, and the inner surface of the channel is lined with barrier-forming endothelial cells. The airway lumen and the vascular channel each extend in a direction along a longitudinal axis of the outer case, wherein the airway lumen and the vascular channel are separated from one another by a portion of the ECM, and wherein the porous membrane allows migration of cells and nutrients from the vascular channel to the airway lumen.

In yet another embodiment, a method of making a Next-Gen Airway-Chip is provided that includes forming an extracellular matrix (ECM) having a cross-sectionally rounded vascular channel, embedding stromal cells within the ECM, lining an inner surface of the vascular channel with barrier-forming endothelial cells, forming an airway lumen having a porous membrane, and providing mucociliated airway epithelial cells on the porous membrane, wherein the airway lumen and the vascular channel are positioned adjacent to one another and each extend in parallel in a longitudinal direction, wherein the airway lumen and the vascular channel are separated from one another by a portion of the ECM, wherein the porous membrane allows migration of cells and nutrients from the vascular channel to the airway lumen.

### BRIEF DESCRIPTION OF THE DRAWINGS

FIG. 1 is a schematic diagram of extracellular matrix (ECM)-embedded vascular channel-on-a-chip according to an exemplary embodiment of a first aspect of the disclosed concept;

FIG. 2 is a flowchart illustrating a method of making an ECM-embedded vascular channel-on-a-chip according to an exemplary embodiment of the disclosed concept;

FIG. 3 is a schematic diagram illustrating certain portions of the method shown in FIG. 2 according to an exemplary embodiment;

FIG. 4 is a schematic diagram of an airway-on-a-chip device according to an exemplary embodiment of a second aspect of the disclosed concept; and

FIG. 5 is a schematic diagram of the airway-on-a-chip device of FIG. 4 showing only portions thereof for illustrative purposes.

### DETAILED DESCRIPTION:

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept will now be described, for purposes of explanation, in connection with numerous specific details in order to provide a thorough understanding of the disclosed concept. It will be evident, however, that the disclosed concept can be practiced without these specific details without departing from the spirit and scope of this innovation.

The disclosed concept, in a first aspect thereof, provides a robust and reliable casting-based method that allows for the creation of cross-sectionally rounded ECM-embedded vascular microlumens of desired length in a controlled manner in an organs-on-chips device in vitro for endothelization and co-culture with stromal cells obtained from an organ of interest. The approach of this aspect of the disclosed concept offers high fidelity and enables reconstitution of simple, yet well-defined, uniform, and smooth vascular channels. Moreover, neither pre-treatment of the hollow tube-forming material nor matrix exposure to other reagents, such as aldehydes or pluronic acid, are needed. The disclosed concept in this first aspect provides a method for effective seeding and full coverage of all inner surfaces of a vascular lumen-on-chip with primary human lung microvascular endothelial cells (hLMVEnCs), along with an optimized protocol for co-culture with primary human lung fibroblasts (hLFs). The disclosed concept in this first aspect recreates physiologically relevant homeostatic ECM rheology using natural (instead of synthetic) polymers, with an ability to emulate diseased (e.g., fibrotic lung) matrix stiffness. The lung is a representative organ; however, the disclosed concept can potentially be applied to any other organ of interest.

In addition, the disclosed concept, in a second related aspect thereof, provides a novel design and method of fabrication for a new Next-Generation Lung Airway-on-a-Chip. This Next-Generation Lung Airway-on-a-Chip incorporates the new ECM-embedded vascular microlumens of the disclosed concept with an airway lumen channel. In addition, it incorporates the ability to use primary cells, partial or fully stem cell-derived cells, and/or cells lines to populate the devices. The Next-Generation Lung Airway-on-a-Chip, as described in detail herein, contains (1) an airway lumen with well-differentiated (that is mucociliated under air-liquid interface (ALI)) airway epithelial cells, (2) an ECM as described herein, (3) ECM-embedded stromal cells (such as lung fibroblasts) as described herein, (4) a cross-sectionally rounded microvascular channel that is cast through the ECM and lined with pulmonary endothelial cells as described herein, and (5) a porous membrane that allows migration of cells and nutrients from the vascular channel to the airway lumen. The porous membrane of this second aspect of the disclosed concept unexpectedly but beneficially becomes curved to form a fully rounded airway lumen. This second aspect of the disclosed concept also offers the ability to include both circulating and tissue-resident immune cells (within the airway lumen, ECM, and/or on the surface of vascular endothelium) for experimentation. This novel, architecturally and cellularly complex, translational microphysiological system is designed to be the most advanced mimicry of human lung conducting airways (e.g., small airways, bronchi, tracheas) that can be applied to reproduce lung pathophysiology in vitro. The system integrates ECM and ECM-embedded stromal cells as seen in vivo with mucociliated airway epithelia, vascular endothelial cells, and circulating immune cells (if any). Such integration is not known or practiced in the prior art.

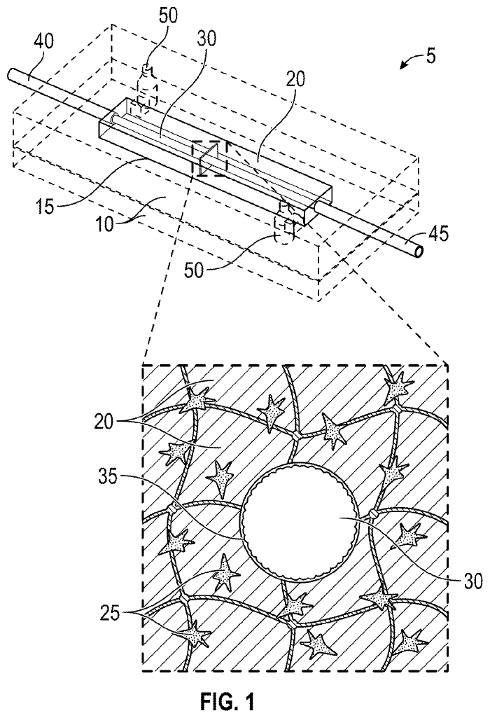

FIG. 1 is a schematic diagram of extracellular matrix (ECM)-embedded vascular channel-on-a-chip 5 according to an exemplary embodiment of the first aspect of disclosed concept. As described in greater detail herein, ECM-embedded vascular channel-on-a-chip 5 comprises a mesoscale long (typically in centimeters), cross-sectionally rounded, three-dimensional microvasculature within a natural extracellular matrix channel on-chip. ECM-embedded vascular channel-on-a-chip 5 in the various embodiments described herein thus allows for accurate recapitulation of pathophysiologically relevant mechanobiology within human blood vessels in an organs on-chips application.

As seen in FIG. 1, ECM-embedded vascular channel-on-a-chip 5 includes a number of outer cases or housings 10 which define an enclosed hollow chamber 15 therein. In the non-limiting exemplary embodiment, the cases 10 include top and bottom slabs as shown in FIG. 1 which are adhered together using, for example, a plasma bonding technique. Also in the non-limiting exemplary embodiment, chamber 15 has a rectangular cross-section as shown. Also in the non-limiting exemplary embodiment, each of the number of cases 10 is made of polydimethylsiloxane (PDMS), although other suitable materials, such as, without limitation, polycarbonate, thermoplastic polymers, or glass, are contemplated within the scope of the disclosed concept. The number of cases 10 may be fabricated by standard soft lithography methods, although other suitable methods are contemplated within the scope of the disclosed concept. As seen in FIG. 1, an extracellular matrix (ECM) 20 is provided within chamber 15. In the illustrated non-limiting exemplary embodiment, ECM 20 has a rectangular cross-section and comprises a hydrogel material. In addition, in the illustrated non-limiting embodiment, a plurality of stromal cells 25 are embedded within ECM 20. In the non-limiting exemplary embodiment, stromal cells 25 include fibroblasts, such as lung fibroblasts.

In addition, a vascular channel 30 is provided within ECM 20 along the longitudinal axis thereof. As seen in the enlarged portion of FIG. 1, vascular channel 30 has a rounded cross-section, such as, without limitation, a circular or oval-shaped cross-section. Furthermore, the inner surface of vascular channel 30 which defines the channel is lined with barrier-forming endothelial cells 35. ECM-embedded vascular channel-on-a-chip 5 further includes an inlet conduit 40 fluidly coupled to the first end of vascular channel 30 through a first side of outer case 10 and an outlet conduit 45 fluidly coupled to the second end of vascular channel 30 through a second, opposite side of outer case 10. ECM-embedded vascular channel-on-a-chip 5 also includes casting ports 50 which are used to form ECM 20 and vascular channel 30 as described in detail elsewhere herein.

Thus, ECM-embedded vascular channel-on-a-chip 5 provides a biodevice in which stromal cells, such as fibroblasts, are embedded within a natural ECM in the perivascular region, thereby mimicking the tunica media and external layers in vasculature or the interstitium between epithelia and endothelia in a given organ. The vascular channel at the center of ECM block provides a uniformly rounded vascular microchannel lined with barrier-forming endothelial cells to emulate tunica intima layer of blood vessels in human bodies.

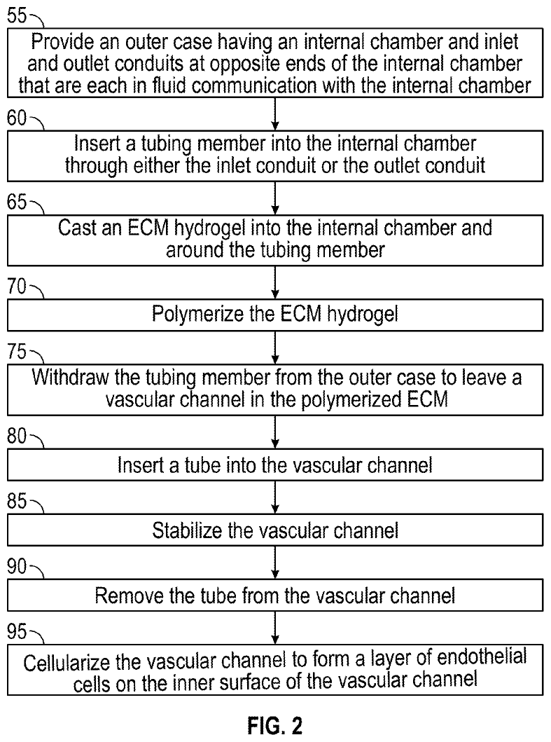

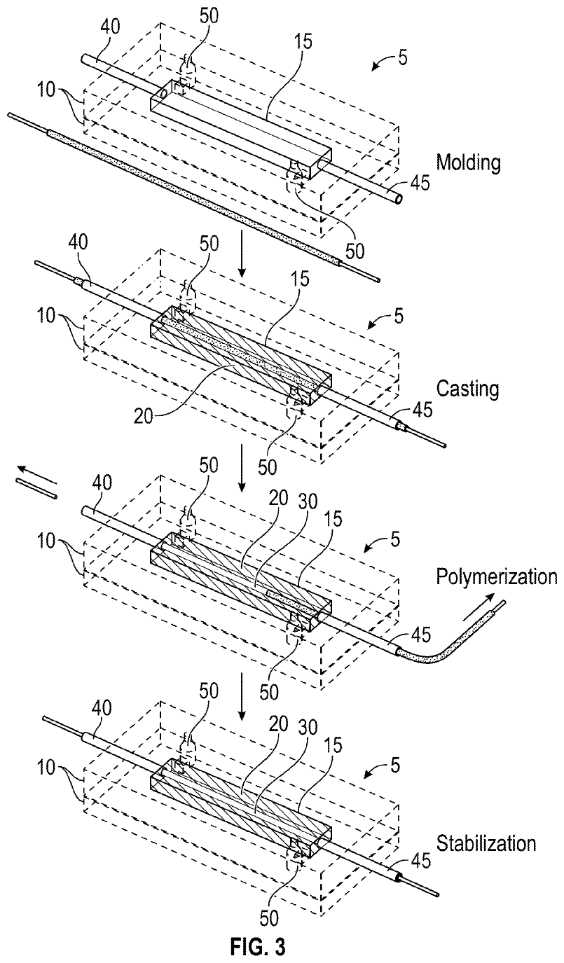

FIG. 2 is a flowchart illustrating a method of making an extra cellular matrix-embedded vascular channel-on-a-chip, such as ECM-embedded vascular channel-on-a-chip 5, according to an exemplary embodiment of the disclosed concept. FIG. 3 is a schematic diagram illustrating certain portions of the method shown and described in FIG. 2 according to an exemplary embodiment.

Referring to FIGS. 2 and 3, the method begins at step 55, wherein an outer case, such as outer case(s) 10 described in connection with FIG. 1, is provided. The outer case includes an internal chamber and inlet and outlet conduits, such as internal chamber 15 and conduits 40, 45 described in connection with FIG. 1. The inlet and outlet conduits are located at opposite ends of the chamber and are in fluid communication with the internal chamber. As noted elsewhere herein, the outer case may be formed by a standard soft lithography method, or another suitable method. Such a method would form the internal chamber and may also provide channels for receiving the inlet and outlet conduits. Alternatively, the inlet and outlet conduits may comprise stainless steel needles that are inserted into the outer case. Next, at step 60, a tubing member is inserted into the internal chamber through either the inlet conduit or the outlet conduit. In the non-limiting exemplary embodiment, the tubing member comprises a portion of silicone tubing with a stainless steel needle at each of its ends, an example of which is shown in FIG. 3.

The method then proceeds to step 65, wherein an ECM hydrogel is cast into the internal chamber and around the tubing member through casting ports provided as part of the outer case, such as ports 50 of case(s) 10 described in connection with FIG. 1. During this step, it is advantageous to prevent air bubbles from being trapped inside the ECM hydrogel. Next, at step 70, the ECM hydrogel is polymerized. In particular, in the exemplary embodiment, the ECM hydrogel is polymerized by incubating it at a particular temperature for a particular period of time, such as 1.5 hours at 37° C. Then, at step 75, the tubing member is withdrawn from the outer case, thereby leaving a vascular channel, such as vascular channel 30, in the polymerized ECM. In the non-limiting exemplary embodiment wherein the tubing member comprises silicone tubing and stainless-steel needles, the tubing is withdrawn in two steps. First, one of the needles is removed from one end of the silicon tubing, and then second, the silicone tubing is slowly pulled out of the outer case from the other end. This is illustrated by the arrows in FIG. 3. Since the silicone tubing of this embodiment is soft, this method of withdrawal prevents damage, such as a collapse or pull-back, to the vascular channel. Another tube is then inserted into the vascular channel at step 80. In the non-limiting exemplary embodiment, the tube comprises a sterilized glass capillary tube as shown in FIG. 3. Thereafter, at step 85, the vascular channel is stabilized. In the exemplary embodiment, step 85 includes dipping the structure into phosphate buffered saline (PBS) or a fibroblast culture medium (Fibro Medium) (in the case where fibroblasts are embedded in the ECM), and incubating the structure overnight (e.g., at 37° C. in a 5% CO₂ incubator) while soaking in Fibro Medium (if used). This stabilization step is advantageous, since without it the internal channel may deform or collapse during the cellularization step that is described below. In addition, use of a glass capillary tube having a diameter that is slightly smaller than the diameter of the vascular channel is particularly advantageous at step 80, as it minimizes damage to the inner channel surfaces following the stabilization and allows the Fibro Medium, if used, to diffuse into the ECM through the gap between the capillary tube and the channel perimeter (thereby supporting survival of the embedded fibroblasts). The tube is then removed from the vascular channel as shown in step 90. Finally, at step 95, the vascular channel is cellularized to form a layer of endothelial cells, such as cells 35 described in connection with FIG. 1, on the inner surface of the vascular channel.

In one particular non-limiting exemplary embodiment, prior to step 65, the internal chamber is treated with polydopamine (PDA) (e.g., 1 mg mL⁻¹) for a predetermined period of time, such as 24 hours. This treatment increases the adhesion of the PDMS used to form the outer case to the ECM hydrogel upon casting. This treatment may use a substance other than PDA as long as it can increase the adhesion of PDMS to the ECM.

In another particular non-limiting exemplary embodiment, the hydrogel used to form the ECM in step 65 consists of 7.5% gelatin, 15 mg mL⁻¹ fibrinogen, 2.5 mM calcium chloride, 1% transglutaminase (TG), and 4 U mL⁻¹ thrombin. To prepare the hydrogel, the gelatin, fibrinogen, calcium chloride, TG, and a phosphate buffered saline (PBS) are mixed together at the desired concentrations, and the mixture is pre-incubated at 37° C. for 45 mins to increase transparency. The PBS can be replaced by a Fibro Medium to support cell viability in the case that stromal cells are to be embedded in the hydrogel. After preincubation, the mixture just described is further mixed with thrombin to form a mixture/solution that is used for the casting described herein. Moreover, the gelatin-fibrinogen-based hydrogels in this embodiment may be replaced by other hydrogels (e.g., synthetic, natural or hybrid), and its composition and theology/stiffness can be adjusted as deemed appropriate (pathophysiologically) or desired by a user.

In another particular non-limiting exemplary embodiment, the cellularization process of step 95 is done using a four-stage seeding protocol as follows. This is not normally obvious for efficient and complete cellularization and required extensive optimization. First, prior to seeding, the structure is connected to a reservoir of Fibro Medium and kept at 37° C., 5% CO₂. In the first round of seeding, hLMVEnCs were harvested and resuspended in an EnC Medium at a density of 15×10⁶ cells mL⁻¹. 40 μl of the cell suspension is then pipetted into the structure comprising the ECM (e.g., ECM 20) and the vascular channel (e.g., vascular channel 30) as described herein via the inlet conduit (e.g., inlet conduit 40). The structure is then incubated inside a 37° C. incubator for 30 mins. While waiting, fresh hLMVEnCs is harvested from another flask and resuspended in the EnC Medium at the same density (15×10⁶ cells mL⁻¹). In the second round of seeding, the structure is rotated 180 degrees before another 40 μl of the freshly harvested cell suspension is pipetted into the structure. The structure is again incubated for 30 mins at 37° C. (while at 180-degree position). The third and four rounds of seeding are done similarly with rotation angles of 90 and 180 degrees, respectively, for cells to adhere to the two sides of the vascular channel. After the four-stage seeding, the channel is vascular flushed with fresh Fibro: EnC Medium supplemented with aprotinin of concentration 20 μg mL⁻¹ and incubated another two hours at 37° C. The structure is then connected to a peristaltic pump to induce a flow of 1 μL min⁻¹ until an endothelial monolayer is observed on-chip, typically after 24 hours. The four-stage seeding protocol is advantageous and particularly useful for vascularizing microchannels on-chip. As EnCs lose their adhesion ability rapidly after seeding to the channel, seeding fresh cells on each surface of the channel will guarantee adequate adhesion as well as the uniformity of the EnC monolayer.

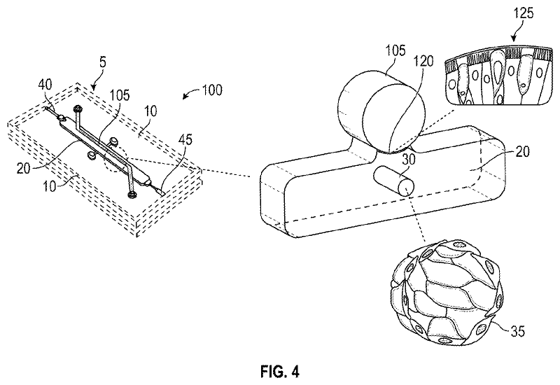

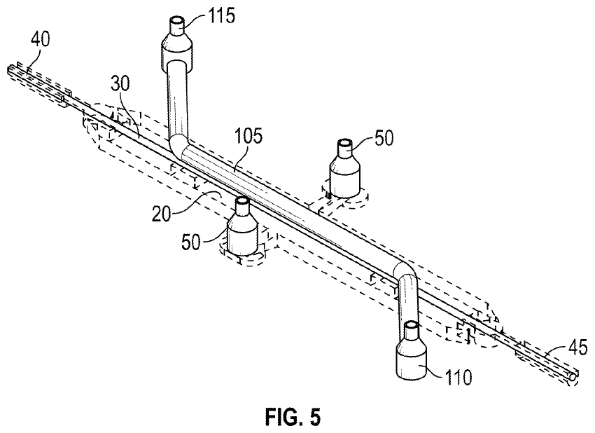

FIG. 4 is a schematic diagram of Next-Gen Airway-Chip device 100 according to an exemplary embodiment of the second aspect of the disclosed concept discussed above. FIG. 5 is a schematic diagram of airway-on-a-chip device 100 showing only portions thereof for illustrative purposes. As seen in FIGS. 4 and 5, airway-on-a-chip device 100 includes a cross-sectionally round airway lumen 105 having an inlet 110 and an outlet 115 that is provided within the case 10 of an embodiment of an ECM-embedded vascular channel-on-a-chip 5 (with ECM embedded stromal cells) as discussed in detail elsewhere herein (like parts are labelled with like reference numerals from FIG. 1). As shown, airway lumen 105 sits atop ECM 20 of ECM-embedded vascular channel-on-a-chip 5, such that airway-on-a-chip device 100 includes two lumens/channels extending in a parallel manner, specifically airway lumen 105 and vascular channel 30.

In the exemplary embodiment, airway lumen 105 is formed within and as part of the top slab of case 10 (i.e., as a hollow channel therein) using soft lithography and PDMS as the material, although other materials and processes may also be used. The bottom portion 120 of airway lumen 105 that is immediately adjacent the top of ECM 20 includes a porous membrane (with micrometer-sized pores) made of PDMS that separates airway channel 105 from ECM 20 having vascular channel 30 formed therein as described. The porous membrane can be rigid or flexible, natural or synthetic, and may be include pores of any desired pore size. Primary human airway epithelial cells (hAEpCs—tracheal, bronchial, or bronchiolar) 125 are cultured and differentiated on top of the PDMS membrane under ALI to construct mucociliated epithelium on-chip, while primary human lung microvascular endothelial cells (hLMVEnCs) 35 are cultured in vascular channel 30 as described elsewhere herein until full confluency to form an endothelial barrier that replicates the lung microvascular capillary seen in vivo. Airway lumen 105 and vascular channel 30 are designed to be parallel and separated by layer of ECM 20 (FIG. 4) with a user-defined thickness (e.g., a few hundred microns) to emulate physiological lung architecture. This setting allows cell-cell interactions between airway lumen 105 and vascular channel 30 as well as the transport of nutrition from vascular channel 30 to support epithelial cells 125 in airway lumen 105. In the exemplary embodiment, both airway lumen 105 and vascular channel 30 have rounded cross-sections, allowing smooth fluid flows inside the channels and simulation of physiological geometries for air and fluid shear experienced by living tissues. Stromal cells, such as fibroblasts and/or airway smooth muscle cells, can be added to ECM 20 in this embodiment in the peri-vascular region. And tissue-resident immune cells such as macrophages and dendritic cells can be added to the epithelial layer or in proximity with other cells in culture.

Finally, according to yet a further aspect of the disclosed concept, it has been found that when using a flexible membrane material (like PDMS) to form airway lumen 105, the channel cross-section of at least a portion of airway lumen 105 (e.g., at least the portion having the porous membrane) will change form semi-circular to circular. In some embodiments, the cross-section of all of airway lumen 105 becomes circular. This was unexpected. While flexible membranes tend to sag to some extent, the level of sag in this instance was found to be more than expected. It is believed that this is due to the combined pull forces from the hydrogel underneath the flexible membrane plus natural bending properties of the membrane. This change to a circular cross-section for at least the porous membrane if not all of lumen 105 is illustrated in FIG. 4.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. An extracellular matrix (ECM)-embedded vascular channel-on-a-chip, comprising:
an outer case having an internal chamber;
an ECM provided within the chamber, the ECM having a cross-sectionally rounded vascular channel having a first end and a second end opposite the first end, wherein an inner surface of the channel is lined with barrier-forming endothelial cells; and
an inlet conduit coupled to the first end of the channel through a first side of the outer case and an outlet conduit coupled to the second end of the channel through a second side of the outer case.

2. The ECM-embedded vascular channel-on-a-chip according to claim 1, wherein the outer case is made of a biocompatible material.

3. The ECM-embedded vascular channel-on-a-chip according to claim 1, wherein the biocompatible material is polydimethylsiloxane (PDMS) or thermoplastic.

4. The ECM-embedded vascular channel-on-a-chip according to claim 1, wherein the chamber and the ECM have a rectangular cross-section.

5. The ECM-embedded vascular channel-on-a-chip according to claim 1, wherein stromal cells are embedded within the ECM.

6. The ECM-embedded vascular channel-on-a-chip according to claim 5, wherein the stromal cells comprise fibroblasts, smooth muscle cells or tissue resident immune cells.

7. The ECM-embedded vascular channel-on-a-chip according to claim 6, wherein the fibroblasts comprise primary human lung fibroblasts (hLFs).

8. The ECM-embedded vascular channel-on-a-chip according to claim 1, wherein the ECM comprises a hydrogel.

9. The ECM-embedded vascular channel-on-a-chip according to claim 8, wherein the hydrogel comprises gelatin, fibrinogen, calcium chloride, transglutaminase (TG), and thrombin in levels selected to mimic a desired pathophysiological setting.

10. The ECM-embedded vascular channel-on-a-chip according to claim 9, wherein the hydrogel comprises 7.5% gelatin, 15 mg mL-1 fibrinogen, 2.5 mM calcium chloride, 1% transglutaminase (TG), and 4 U mL-1 thrombin.

11. The ECM-embedded vascular channel-on-a-chip according to claim 1, wherein the endothelial cells comprise primary human lung microvascular endothelial cells (hLMVEnCs), other primary human or animal endothelial cells, or stem cell-derived endothelial cells.

12. The ECM-embedded vascular channel-on-a-chip according to claim 1, wherein the outer case includes a number of casting ports fluidly coupled to the chamber.

13. The ECM-embedded vascular channel-on-a-chip according to claim 5, wherein at least one of the endothelial cells or the stromal cells are stem cell derived cells.

14. A method of making an extracellular matrix (ECM)-embedded vascular channel-on-a-chip, comprising:
providing an outer case having an internal chamber;
providing an inlet conduit through a first side of the outer case and providing an outlet conduit through a second side of the outer case, the inlet conduit and the outlet conduit each being in fluid communication with the chamber;
inserting a tubing member into the chamber through at least one of the inlet conduit and the outlet conduit;
casting an ECM hydrogel into the chamber and around the tubing member through a casting port provided in the outer case;
polymerizing the ECM hydrogel within the chamber;
withdrawing the tubing member from the outer case to leave a vascular channel in the polymerized ECM hydrogel;
inserting a tube into the vascular channel;
stabilizing the vascular channel with the inserted tube;
removing the tube from the vascular channel; and
cellularizing the vascular channel to form a layer of endothelial cells on an inner surface of the channel.

15. The method according to claim 14, wherein the tube is a glass capillary tube.

16. The method according to claim 14, wherein the tubing member is a silicone tube having a needle at each end thereof.

17. The method according to claim 11, wherein the outer case is made of polydimethylsiloxane (PDMS) or any other biocompatible material, and wherein prior to the casting, the internal chamber is treated with polydopamine (PDA) or another bio-adhesive of interest.

18. The method according to claim 14, wherein stromal cells are embedded within the ECM hydrogel.

19. The method according to claim 14, wherein the stromal cells comprise fibroblasts, smooth muscle cells or tissue resident immune cells.

20. The method according to claim 14, wherein the ECM hydrogel comprises gelatin, fibrinogen, calcium chloride, transglutaminase (TG), and thrombin in levels selected to mimic a desired pathophysiological setting.

21. The method according to claim 20, wherein the hydrogel comprises 7.5% gelatin, 15 mg mL-1 fibrinogen, 2.5 mM calcium chloride, 1% transglutaminase (TG), and 4 U mL-1 thrombin.

22. The method according to claim 14, wherein the cellularizing comprises a four-stage seeding protocol wherein the vascular channel is rotated through 360° and includes incubation after each stage of the seeding protocol.

23. An airway-on-a-chip, comprising:
an outer case having an internal chamber:
an airway lumen provided within the outer case, wherein the airway lumen includes a porous membrane, and wherein mucociliated airway epithelial cells are provided on the porous membrane; and
an extracellular matrix (ECM) provided within the chamber, the ECM having a cross-sectionally rounded vascular channel, wherein stromal cells are embedded within the ECM, wherein an inner surface of the vascular channel is lined with barrier-forming endothelial cells, wherein the airway lumen and the vascular channel each extend in a direction along a longitudinal axis of the outer case, wherein the airway lumen and the vascular channel are separated from one another by a portion of the ECM, and wherein the porous membrane allows migration of cells and nutrients from the vascular channel to the airway lumen.

24. The airway-on-a-chip according to claim 23, wherein the porous member forms a part of a bottom of the airway lumen adjacent to the portion of the ECM.

25. The airway-on-a-chip according to claim 23, wherein the airway lumen has a rounded or semi-rounded cross-section.

26. The airway-on-a-chip according to claim 23, wherein the outer case is made of polydimethylsiloxane (PDMS) or any other biocompatible material.

27. The airway-on-a-chip according to claim 23, wherein the chamber and the ECM have a rectangular cross-section.

28. The airway-on-a-chip according to claim 23, wherein the stromal cells comprise fibroblasts, smooth muscle cells or tissue resident immune cells.

29. The airway-on-a-chip according to claim 28, wherein the fibroblasts comprise primary human lung fibroblasts (hLFs).

30. The airway-on-a-chip according to claim 23, wherein the ECM comprises a hydrogel.

31. The airway-on-a-chip according to claim 30, wherein the hydrogel comprises gelatin, fibrinogen, calcium chloride, transglutaminase (TG), and thrombin in levels selected to mimic a desired pathophysiological setting.

32. The airway-on-a-chip according to claim 31, wherein the hydrogel comprises 7.5% gelatin, 15 mg mL-1 fibrinogen, 2.5 mM calcium chloride, 1% transglutaminase (TG), and 4 U mL-1 thrombin.

33. The airway-on-a-chip according to claim 23, wherein the endothelial cells comprise primary human lung microvascular endothelial cells (hLMVEnCs), other primary human or animal endothelial cells, or stem cell-derived endothelial cells.

34. The airway-on-a-chip according to claim 23, wherein the epithelial cells comprise primary human airway epithelial cells (hAEpCs).

35. The airway-on-a-chip according to claim 23, wherein at least one of the epithelial cells, the endothelial cells or the stromal cells are stem cell derived cells.

36. The airway-on-a-chip according to claim 24, wherein at least the porous membrane has a circular cross-section.

37. The airway-on-a-chip according to claim 36, wherein the portion of the ECM sags to have a circular cross-section to accommodate the porous membrane.

38. The airway-on-a-chip according to claim 36, wherein the airway lumen has a circular cross-section.

39. A method of making an airway-on-a-chip, comprising:
forming an extracellular matrix (ECM) having a cross-sectionally rounded vascular channel:
embedding stromal cells within the ECM;
lining an inner surface of the vascular channel with barrier-forming endothelial cells;
forming an airway lumen having a porous membrane; and
providing mucociliated airway epithelial cells on the porous membrane;
wherein the airway lumen and the vascular channel are adjacent to one another and each extend in parallel in a longitudinal direction, wherein the airway lumen and the vascular channel are separated from one another by a portion of the ECM, and wherein the porous membrane allows migration of cells and nutrients from the vascular channel to the airway lumen.

40. The method according to claim 39, wherein the porous member forms a part of a bottom of the airway lumen adjacent to the portion of the ECM.

41. The method according to claim 39, wherein the airway lumen has a rounded or semi-rounded cross-section.

42. The method according to claim 39, wherein the ECM has a rectangular cross-section.

43. The method according to claim 39, wherein the stromal cells comprise fibroblasts, smooth muscle cells or tissue resident immune cells.

44. The method according to claim 43, wherein the fibroblasts comprise primary human lung fibroblasts (hLFs).

45. The method according to claim 39, wherein the ECM comprises a hydrogel.

46. The method according to claim 45, wherein the hydrogel comprises gelatin, fibrinogen, calcium chloride, transglutaminase (TG), and thrombin in levels selected to mimic a desired pathophysiological setting.

47. The method according to claim 46, wherein the hydrogel comprises 7.5% gelatin, 15 mg mL-1 fibrinogen, 2.5 mM calcium chloride, 1% transglutaminase (TG), and 4 U mL-1 thrombin.

48. The method according to claim 39, wherein the endothelial cells comprise primary human lung microvascular endothelial cells (hLMVEnCs), other primary human or animal endothelial cells, or stem cell-derived endothelial cells.

49. The method according to claim 39, wherein the epithelial cells comprise primary human airway epithelial cells (hAEpCs).

50. The method according to claim 39, wherein at least one of the epithelial cells, the endothelial cells or the stromal cells are stem cell derived cells.

51. The method according to claim 40, wherein at least the porous membrane has a circular cross-section.

52. The method according to claim 51, wherein the portion of the ECM sags to have a circular cross-section to accommodate the porous membrane.

53. The method according to claim 51, wherein the airway lumen has a circular cross-section.
